(19) United States
(12) Patent Application Publication
(10) Pub. No.: US 2005/0182459 A1
(43) Pub. Date: Aug. 18, 2005

(54) Apparatus for harvesting and storing energy on a chip

(76) Inventors: Constance M. John, San Francisco, CA (US); Varghese John, San Francisco, CA (US)

Correspondence Address: Douglas E. Mackenzie, Suite 302, 2645 Ocean Ave., San Francisco, CA, 94132, US

(21) Appl. No.: 11/027,335

(22) Filed: Dec. 29, 2004

Related U.S. Application Data
(60) Provisional application No. 60/533,449, filed on Dec. 30, 2003.

(51) Int. Cl. A61N 1/08

(52) U.S. Cl. 607/61

(57) ABSTRACT

An apparatus for receiving energy through space and storing the received energy on a chip includes an antenna for receiving the energy, a converter circuit for converting the received energy, and a storage device for storing the converted energy, the antenna, converter circuit and storage device being formed on the chip. Associated methods of use of the apparatus are also disclosed.

### CROSS REFERENCE TO RELATED APPLICATIONS

The present invention claims priority under 35 U.S.C. 119(e) from provisional patent application Ser. No. 60/533,449, entitled “Applications of Energy Harvesting System on a Chip (EHSOC)”, filed on Dec. 30, 2003, the disclosure of which is herein incorporated by reference in its entirety.

### BACKGROUND OF THE INVENTION

The present invention generally relates to energy harvesting and more particularly to apparatus for harvesting and storing radio frequency (RF) energy on a semiconductor chip at a miniaturized scale such as micro or nano scale and uses thereof.

It is known in the prior art to transmit energy through space from a base station to a remote station. One such system is disclosed in U.S. Pat. No. 6,289,237 entitled “Apparatus for Energizing a Remote Station and Related Method”. The disclosed base station transmits energy which may be RF power, light, acoustic, magnetic or other suitable forms of space transmitted or “radiant” energy to the remote station. Within the remote station, the received energy is converted into DC power which serves to operate the remote station. The source of power for the remote station is the base station and, therefore, there is no need for the remote station to carry an electrical storage device such as a battery. It is suggested that this facilitates the remote station being encapsulated within a suitable protective material, such as a resinous plastic. Homopolymers, elastomers and silicon dioxide are also suggested as suitable materials for such purposes. Further, it is suggested that this facilitates miniaturization of the remote station and placing the remote station in functionally desirable locations which need not be readily accessible. The remote station, for example, could be implanted in a patient.

The use of a wireless communication link between a base station and transponders in a radio frequency identification system employing modulated back-scattered waves is also known. See Rao, An Overview of Bulk Scattered Radio Frequency Identification System (RFID) IEEE (1999). It has also been suggested to employ a silicon chip in a transponder having a change pump or voltage doubler current. Hornby, RFID Solutions for the Express Parcel and Airline Baggage Industry, Texas Instruments, Limited (Oct. 7, 1999).

For use in miniaturized electronic chip systems, an electronic article containing a microchip having at least one antenna structured to communicate with an antenna remotely disposed with respect to the microchip is disclosed in U.S. Pat. No. 6,615,074 entitled “Apparatus for Energizing a Remote Station and Related Method”. With reference to FIG. 1, there is shown the disclosed antenna layout for use on an electronic microchip. FIG. 2 shows the disclosed fabricated die containing an on-board antenna. Power enhancement is achieved using a voltage doubler such as shown in FIG. 3. The antenna of the disclosed apparatus is comparable in volume to the Smart Dust device as shown in FIG. 4. Smart Dust is a combination MEMS/Electronic device on the order of 1 mm×1 mm×1 mm.

In spite of the foregoing known systems and apparatus, none describe an apparatus for harvesting RF energy and storing the harvested RF energy for long term use in a variety of applications including miniaturized scale applications such as micro and nanotechnology and for implanted biomedical devices.

### SUMMARY OF THE INVENTION

In accordance with one aspect of the invention, an apparatus for receiving energy through space and storing the received energy on a chip includes an antenna for receiving the energy, a converter circuit for converting the received energy, and a storage device for storing the converted energy, the antenna, converter circuit and storage device being formed on the chip.

In accordance with another aspect of the invention, a method of treating a disease includes the steps of providing an apparatus having an antenna for receiving energy through space, a converter circuit for converting the received energy, a storage device for storing the converted energy, the antenna, converter circuit, and storage device being formed on a chip, coupling the apparatus to a drug delivery system, and powering the drug delivery system from the apparatus.

In accordance with yet another aspect of the invention, a method of electrically stimulating a tissue includes the steps of providing an apparatus having an antenna for receiving energy through space, a converter circuit for converting the received energy, a storage device for storing the converted energy, the antenna, converter circuit, and storage device being formed on a chip, coupling the apparatus to an electrode, and powering the electrode from the apparatus.

In accordance with another aspect of the invention, a method of treating a disease includes the steps of harvesting RF energy, storing the harvested RF energy on a chip, and using the stored energy to treat the disease.

There has been outlined, rather broadly, the more important features of the invention in order that the detailed description thereof that follows may be better understood, and in order that the present contribution to the art may be better appreciated. There are, of course, additional features of the invention that will be described below and which will form the subject matter of the claims appended herein.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a plan view of a prior art antenna layout;

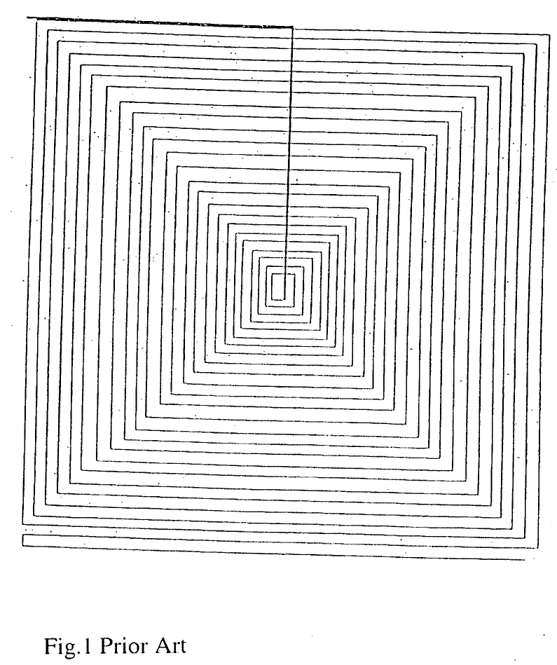

FIG. 2 is a plan view of a prior art fabricated die containing an on-board antenna;

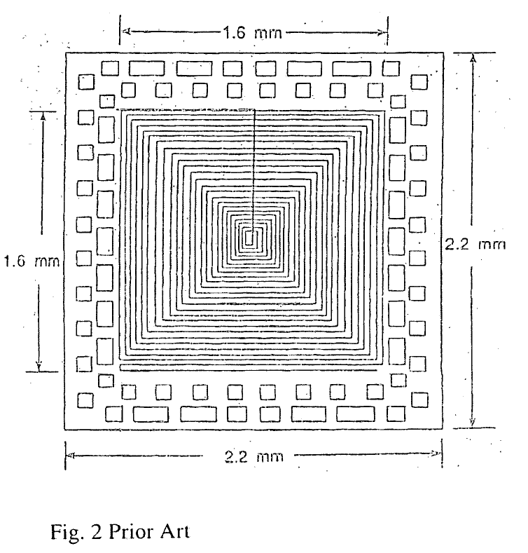

FIG. 3 is a schematic illustration of a prior art voltage doubler circuit;

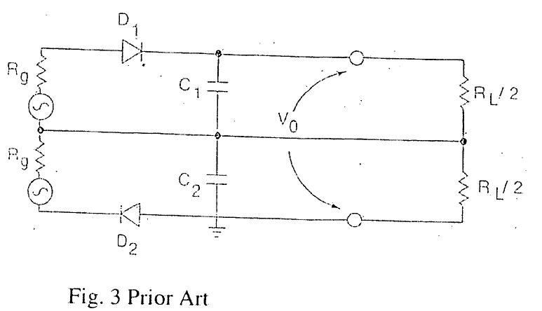

FIG. 4 is a prior art graph of power as a function of antenna volume;

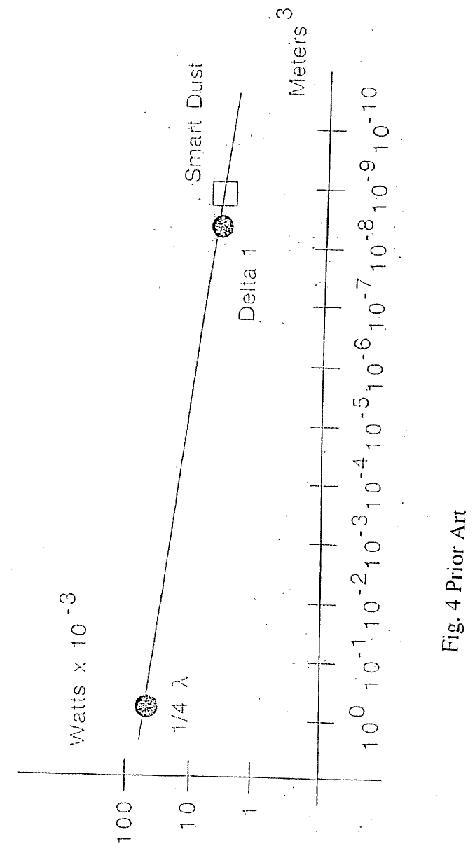

FIG. 5 is a schematic representation of an apparatus for harvesting and storing energy on a chip coupled to a micropump/PZT device in accordance with the present invention;

FIG. 6 is a schematic representation of the apparatus in use in accordance with the present invention;

FIG. 7 is a schematic representation of the apparatus in an alternative use in accordance with the present invention;

FIG. 8 is a schematic representation of the apparatus in an alternative use in accordance with the present invention;

FIG. 9 is a schematic representation of the apparatus in an alternative use in accordance with the present invention;

FIG. 10 is a schematic representation of the apparatus in an alternative use in accordance with the present invention;

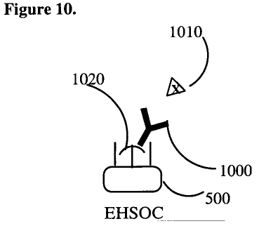

FIG. 11 is a schematic representation of the apparatus in an alternative use in accordance with the present invention;

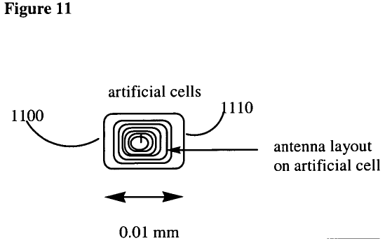

FIG. 12 is a schematic representation of the apparatus in an alternative use in accordance with the present invention;

FIG. 13 is a schematic representation of the apparatus in an alternative use in accordance with the present invention;

FIG. 14 is a schematic representation of the apparatus in an alternative use in accordance with the present invention;

FIG. 15 is a schematic representation of the apparatus in an alternative use in accordance with the present invention;

FIG. 16 is a schematic representation of the apparatus in an alternative use in accordance with the present invention;

FIG. 17 is a schematic representation of the apparatus in an alternative use in accordance with the present invention;

FIG. 18 is a schematic representation of the apparatus in an alternative use in accordance with the present invention;

FIG. 19 is a schematic representation of the apparatus in an alternative use in accordance with the present invention;

FIG. 20 is a schematic representation of the apparatus in an alternative use in accordance with the present invention; and

FIG. 21 is a schematic representation of the apparatus in an alternative use in accordance with the present invention.

### DETAILED DESCRIPTION OF THE INVENTION

The present invention provides an apparatus for harvesting and storing energy on a chip. The energy source for the apparatus may include radio frequency (RF) energy, either ambient RF energy or RF energy transmitted to the apparatus by a dedicated source using microwave energy, laser energy, sound energy, nuclear energy, ocean wave energy, deep space energy and wind energy.

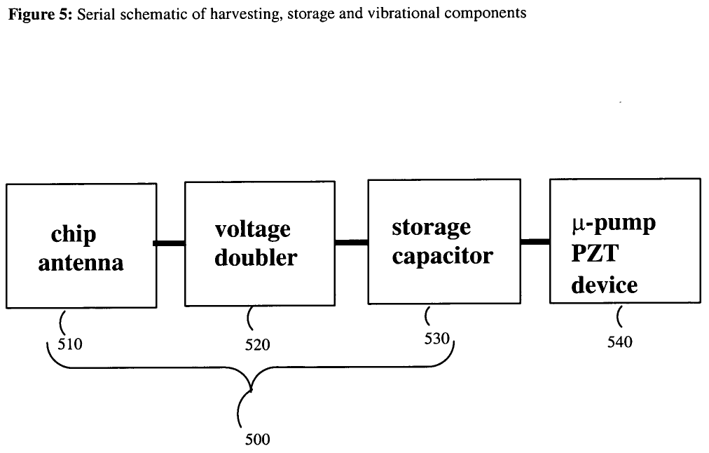

With reference to FIG. 5 an apparatus generally designated 500 in accordance with the invention may include an antenna 510 coupled to a voltage doubler 520 formed on a semiconductor chip (not shown). Antenna 510 may include an antenna having an effective antenna size greater than a physical antenna size for use on the chip. Voltage doubler 520 may provide a means for getting a high DC output voltage from a remote AC source. A storage device 530 may be coupled to the voltage doubler 520 to provide a means for storing the output voltage. Storage device 530 may include a capacitor. In an exemplary application, a micro-pump 540 may be powered by the apparatus 500.

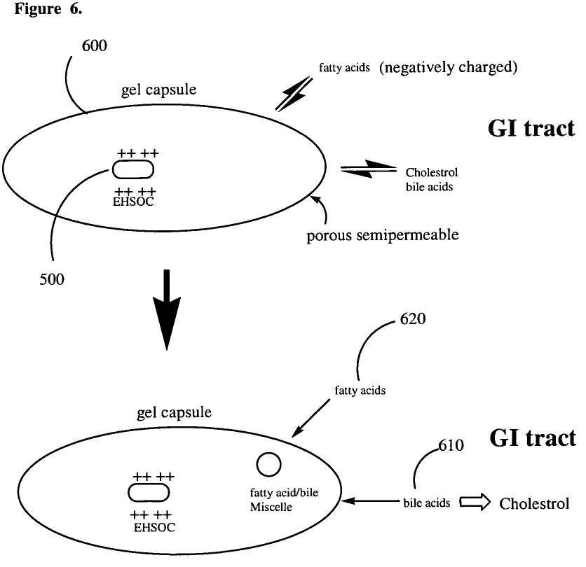

In use, the apparatus 500 may be encapsulated by a gel capsule 600 as shown in FIG. 6. Apparatus 500 may include a charge carrying means for carrying a positive charge to provide adsorption of cholesterol 610 and fatty acids 620 from a patient's gastrointestinal (GI) tract into the gel capsule 600. In this manner a plasma cholesterol level of a patient may be lowered. The reduction in plasma fatty acids may also find application in weight reduction practices. Those skilled in the art will appreciate that the apparatus 500 may alternatively carry a negative charge to provide adsorption of positively charged ions and molecules.

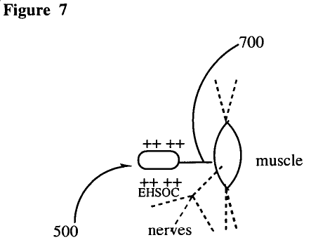

With reference to FIG. 7, the apparatus 500 may include an electrode 700 for providing electrical stimulation. In use the apparatus 500 having the electrode 700 may be used to provide electrical stimulation to muscles and nerves.

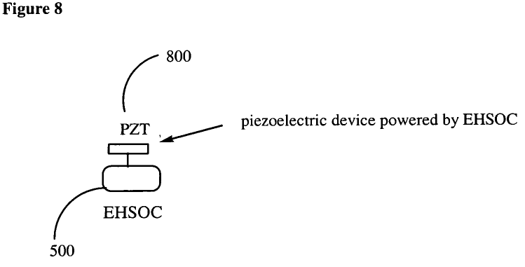

A piezoelectric device 800 as shown in FIG. 8 may be coupled to the apparatus 500 to provide for vibration and/or heating. Piezoelectric device 800 may provide vibrational stimulation to muscles and tissues.

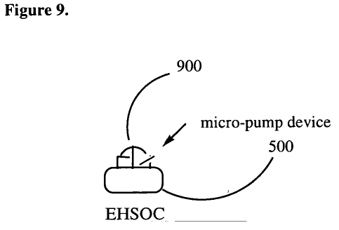

With reference to FIG. 9, a pump 900 may be coupled to the apparatus 500 to provide for pumping of drugs and other materials.

A device for detecting the binding of a molecule 1010 to an antibody 1000 may include a signaling means having moveable member 1020 coupled to the apparatus 500. Movement of the moveable member 1020 may provide a signal to the apparatus 500 of the binding of the molecule 1010.

An artificial cell 1100 may include the apparatus 500 implanted in a cell 1110. The artificial cell 1100 may include synthetic cells similar to bacteria that are not truly alive.

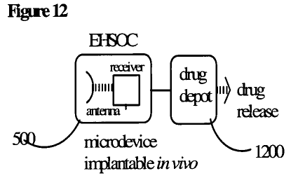

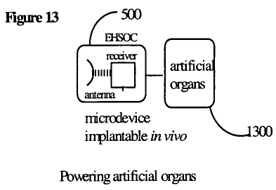

With reference to FIG. 12, the apparatus 500 may be coupled to a drug delivery device 1200. With reference to FIG. 13, the apparatus 500 may be coupled to an artificial organ 1300.

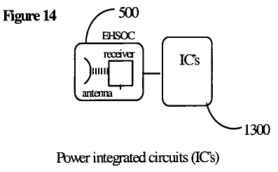

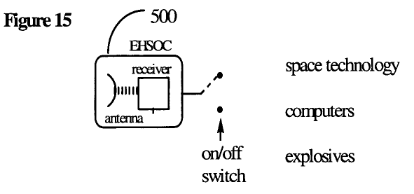

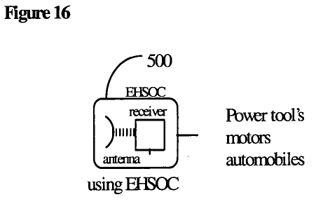

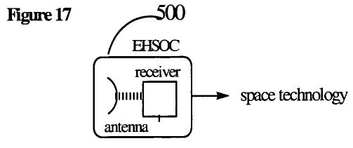

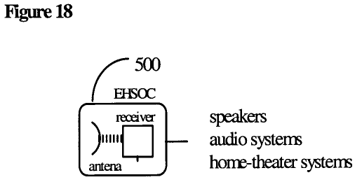

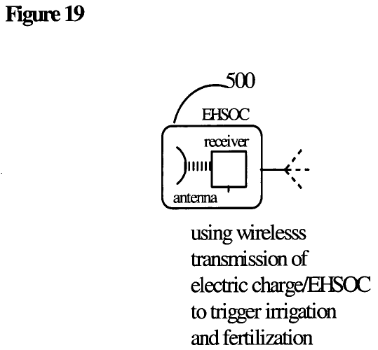

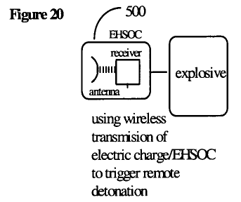

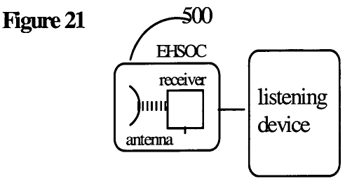

The apparatus 500 may be used to power a variety of devices outside of a body. Examples are shown in FIG. 14 for use in computer devices, FIG. 15 for use as a switching means in space technology, computers, and explosives, FIG. 16 for use in power tools, motors, and automobiles, FIG. 17 for use in space technology, FIG. 18 for use in speakers, audio systems, and home theatre systems, FIG. 19 for use in irrigation and fertilization triggered by a signal from the apparatus 500, FIG. 20 for use in triggering explosives, and FIG. 21 for use in remote listening devices.

The present invention advantageously provides for an apparatus for harvesting and storing energy on a chip. The apparatus may be used as a power source for a plurality of devices, a signaling means, a switching means and a charge carrying means. The apparatus may be implanted in or administered to a human or an animal for treating or preventing a disease including Parkinson's disease, dystonia, epilepsy, depression, Alzheimer's disease, Huntington's disease, Prion disease, down's syndrome, autism, Jacob Creutzfeldt disease, neuropathic and back pain, cancer, stroke related movement disorders, urinary incontinence, cardiovascular disease, and chronic headache.

The present invention further provides for a container kit including a plurality of containers, each container having a unit dose of the apparatus of the invention. Each container may include a dose for oral delivery and include a tablet, a gel and a capsule having the apparatus of the invention. Containers may be adapted for parenteral delivery and include a depot product, a syringe, an ampoule and a vial having the apparatus of the invention. Containers may further be adapted for topical delivery and include a patch, a medipad, an ointment and a cream having the apparatus of the invention.

With regard to these diseases, the term “treating” means that devices of the invention can be used in humans with existing disease. The devices of the invention will not necessarily cure the patient who has the disease but will delay or slow the progression or prevent further progression of the disease thereby giving the individual a more useful life span.

The term “preventing” means that that if the devices of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease. By delaying the onset of the disease, devices of the invention will have prevented the individual from getting the disease during the period in which the individual would normally have gotten the disease or reduce the rate of development of the disease or some of its effects but for the administration of devices of the invention up to the time the individual ultimately gets the disease. Preventing also includes administration of the devices of the invention to those individuals thought to be predisposed to the disease.

In a preferred aspect, the devices of the invention are useful for slowing the progression of disease symptoms.

In another preferred aspect, the devices of the invention are useful for preventing the further progression of disease symptoms.

In treating or preventing the above diseases, the devices of the invention are administered in a therapeutically effective amount. The therapeutically effective amount will vary depending on the particular device used and the route of administration, as is known to those skilled in the art.

In treating a patient displaying any of the diagnosed above conditions a physician may administer a device of the invention immediately and continue administration indefinitely, as needed.

The many features and advantages of the invention are apparent from the detailed specification, and thus, it is intended by the appended claims to cover all such features and advantages which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. An apparatus for receiving energy through space and storing the received energy on a chip comprising:
an antenna for receiving the energy;
a converter circuit for converting the received energy; and
a storage device for storing the converted energy, the antenna, converter circuit, and storage device being formed on the chip.

2. The apparatus of claim 1, wherein the antenna has an effective antenna size greater than a physical antenna size.

3. The apparatus of claim 1, wherein the converter circuit comprises a voltage doubler.

4. The apparatus of claim 1, wherein the storage device comprises a capacitor.

5. The apparatus of claim 1, further comprising a signaling means powered by the stored energy.

6. The apparatus of claim 1, further comprising a charge carrying means powered by the stored energy.

7. The apparatus of claim 1, further comprising a switching means powered by the stored energy.

8. The apparatus of claim 1, further comprising a vibrational means powered by the stored energy.

9. The apparatus of claim 1, further comprising a heating means powered by the stored energy.

10. The apparatus of claim 1, further comprising a cooling means powered by the stored energy.

11. The apparatus of claim 1, wherein the chip has dimensions in the micrometer range.

12. The apparatus of claim 1, wherein the chip has dimensions in the nanometer range.

13. A method of treating a disease comprising the steps of:
providing an apparatus having an antenna for receiving energy through space, a converter circuit for converting the received energy, a storage device for storing the converted energy, the antenna, converter circuit, and storage device being formed on a chip;
coupling the apparatus to a drug delivery system; and
powering the drug delivery system from the apparatus.

14. A method of electrically stimulating a tissue comprising the steps of:
providing an apparatus having an antenna for receiving energy through space, a converter circuit for converting the received energy, a storage device for storing the converted energy, the antenna, converter circuit, and storage device being formed on a chip;
coupling the apparatus to an electrode; and
powering the electrode from the apparatus.

15. A method of treating a disease comprising the steps of:
harvesting RF energy;
storing the harvested RF energy on a chip; and
using the stored energy to treat the disease.

16. The method of claim 15, wherein harvesting RF energy comprises providing an apparatus having an antenna for receiving RF energy through space and a converter circuit for converting the received RF energy.

17. The method of claim 16, wherein storing the harvested RF energy on a chip comprises providing a storage device for storing the converted RF energy, the antenna, converter circuit, and storage device being formed on the chip.

18. The method of claim 15, wherein using the stored energy to treat the disease comprises powering a drug delivery system.

19. The method of claim 15, wherein using the stored energy to treat the disease comprises charging a device disposed within a gel capsule.

20. The method of claim 15, wherein using the stored energy to treat the disease comprises providing an electrical stimulation.

21. The method of claim 15, wherein using the stored energy to treat the disease comprises providing a mechanical stimulation.

22. The method of claim 15, wherein using the stored energy to treat the disease comprises powering a micro pump.

23. The method of claim 15, wherein using the stored energy to treat the disease comprises detecting the binding of a molecule of interest.

24. The method of claim 15, wherein using the stored energy to treat the disease comprises powering an artificial cell.

25. The method of claim 15, wherein using the stored energy to treat the disease comprises powering an artificial organ.

26. A method of preventing a disease comprising the steps of:
harvesting RF energy;
storing the harvested RF energy on a chip; and
using the stored energy to prevent the disease.
